(19) United States
(12) Patent Application Publication
(10) Pub. No.: US 2022/0409733 A1
(43) Pub. Date: Dec. 29, 2022

(54) COMPOSITIONS AND METHODS FOR TREATING CANCER

(71) Applicant: Roohangiz SAFAEI, San Diego, CA (US)

(72) Inventor: Roohangiz SAFAEI, San Diego, CA (US)

(21) Appl. No.: 17/429,311

(22) PCT Filed: Feb. 6, 2020
(86) PCT No.: PCT/US2020/017096

§ 371 Date: Aug. 6, 2021

Related U.S. Application Data
(60) Provisional application No. 62/802,347, filed on Feb. 7, 2019.

(51) Int. Cl. A61K 47/64; A61P 35/00

(52) U.S. Cl. CPC A61K 47/6425; A61P 35/00

(57) ABSTRACT

Provided herein are compositions comprising a DR6 peptide; and methods for treating cancer and/or a tumor, including a platinum drug resistant tumor or cancer, in a patient in need thereof.

### BACKGROUND

### Field of the Invention

The invention generally relates to compositions and methods for treating cancer.

### Related Art

Ovarian cancer is the highest-ranking cause of death among gynecological cancers. Late stage diagnosis due to the inadequate imaging and screening technologies [3] and development of resistance to the platinum (Pt) drugs Cisplatin (cDDP) and carboplatin (CBDCA) are the main causes of failure of treatment in many ovarian cancers. At present very few second line options are available for the treatment and reversal of the acquired resistance to the Pt drugs [4, 5].

Mechanisms that mediate Pt drug resistance are many, most notably DNA repair mechanism [6-11], drug accumulation mechanisms [12], and metal homeostasis mechanisms [13-16]. Transport defects are the most frequently reported in Pt drug resistant tumors and appear to be related to cross-resistance between the Pt drugs, metals and metalloids such as copper (Cu) [17-19], whose uptake and efflux closely mirror those of the Pt drugs in most cells [14, 18-21]. The homeostasis of iron (Fe) is also dysregulated in many Pt resistant tumors [22] that frequently exhibit increased uptake and storage of Fe through upregulation of transferrin (TF), transferrin receptor (TRF1) and ferritins (FTL and FTH1) and downregulation of the efflux protein FPN1 [15, 23-26]. The role of transition metals in Pt resistance is highly complex. Transition metals control many vital processes through their roles as electron donors and acceptors including respiration, energy production, redox homeostasis [27-29], DNA replication [30, 31], transcription [32-34] and signaling [35-46]. Targeting transition metals through mechanisms that mediate their transporter [27] or bioavailability [16, 24, 47-50] has had mixed effects does not seem to be a long term cancer treatment option.

Use of multiple agents with diverse molecular targets [51, 52] have also been proposed for the treatment of Pt drug resistant tumors; such options have been made possible through innovations in targeted drug delivery with composite particles that can carry multiple therapeutics and detection agents for controlled or sustained release at tumor site, while reducing damage to non-target tissues, and minimizing side effects [53].

Therefore, what is needed is a system and method that overcomes these significant problems found in the conventional systems as described above.

### SUMMARY

To address the need for specific diagnostic and treatment tools, we have developed a targeting probe, ARPNS (APP reactive nanoprobes) probe, that can identify and deliver detection and treatment agents directly to tumors, e.g., the Pt drug resistant tumors, and the like.

Provided herein are safe and side-effect-free carriers for delivery of platinum containing therapeutic products and other anticancer drugs for treating cancer, such as ovarian cancer, and the like. Provided her are tumor specific carriers for delivery of imaging agents for detection of tumors such as ovarian cancer, and the like, in vivo and in vitro. Provided herein are tumor-cell and pathway specific biomarkers for targeting FDA approved Pt complexes and other anticancer agents for targeted delivery to tumor cells. In accordance with the present invention, ADR6NI has been found to be an excellent probe for this purpose; it can add specificity, increase potency, and reduce the non-specific toxicity of Pt containing and many other anticancer drugs. It can be used for detection of tumors with specificity and accuracy. Also provided herein are ADR6NI drug testing assays.

In accordance with the present invention, methods and related compositions for detecting, preventing, reversing and inhibiting Pt drug resistance in ovarian tumors are provided herein as a result of identifying novel biomarkers that can be manipulated for targeting and drug delivery for these particular tumors and cancers. Our search for Pt drug resistance biomarkers has led to the identification of a class of molecules that are known for their role in Alzheimer's disease. We detected an increase in the expression of genes encoding the family of amyloid precursor proteins, APP, APLP1 and APLP2. The augmented expression of these molecules is both at the mRNA and protein level is consistent with the findings of other laboratories that report over-expression of these proteins in Pt resistant cancers [54-56] and in a wide range of malignancies including those of the pancreatic [57-59], colon [60], breast [61, 62], prostate [63-65], lung [66-68], thyroid [69], and testicular [70-73] tissues, leukemias [74], lymphomas [75], squamous cell carcinomas [76], nasopharyngeal carcinomas [77], and oral squamous cell carcinomas [78]. Analysis of mRNA from more than 10 isogenic pairs of ovarian and head and neck carcinoma cell lines, sensitive/resistant to cisplatin, carboplatin, and oxaliplatin showed that the resistant cells expressed at least one or more members of the APP family [20, 79]. Protein analysis of three pairs of cDDP resistant/sensitive ovarian carcinoma cell lines, 2008 vs. 2008/C13*5.25, IGROV1 vs. IGROV1-CP and A2780 vs. A2780-CP showed that the resistant cells overexpressed all three of the APP, APLP1 and APLP2 protein, when analyzed by Western blotting (FIG. 1A).

The functional significance of the APP family to the state of Pt drug resistance was demonstrated in experiments in which the expression of APP was downregulated by using antisense morpholino oligonucleotides (ONS) in the Pt resistant subline A2780-CP. Overnight treatment of A2780-CP cells with 25 μM APP ONS blocked the expression of APP and sensitized cells to the toxic effects of cDDP as measured during a 1 drug exposure time by CCK-8 assay (FIG. 1B). The IC50 values of the A2780-CP cells following APP downregulation had dropped from 63.19±1.9 μM to 12.14±0.3 μM (p<0.01), a level that was similar to the IC50 values measured for the parental A2780 cell line (10.16±0.32, p≤0.6). Scrambled oligonucleotides failed to alter the sensitivity of the A2780-CP cells to cDDP (FIG. 1B).

Although the exact mechanism by which the APP family members control Pt drug resistance remains to be determined, effect on Fe regulatory networks is likely to play a significant role. APP family members are known to promote angiogenesis, metastasis [59, 63, 80-85] and proliferation [63, 86, 87], mainly through a soluble form (sAPPα) produced by shedding of their ectodomains by α-secretase enzymes that function in the non-amyloidogenic pathway [88, 89]. The trophic effects of sAPPα involve the Fe homeostasis mechanism [90], linking the APP family to the network of transition metals regulatory proteins.

The APP family is believed to play different roles in cancer from those in the Alzheimer's disease, as evidenced from preference for non-amyloidogenic processing in and participation in proliferative and trophic pathway in cancer cells [82, 83, 91] as well as production of unique, cancer specific variants through differential mRNA splicing. The expression of the APP variant, APP751, with inclusion of exon 7 and exclusion of exon 8 has been shown in cancers of breast [92], lung, colon [67], and ovaries [84, 93]. Similarly, cancer-specific splice variants of APLP2, mainly APLP2751, have been shown in breast cancer cells [84].

The APP751 variant mRNA is the dominant form in the three Pt resistant ovarian cell lines, 2008/C13*5.3, IGROV1-CP and A2780-CP that as examined by RT-PCR using oligonucleotides specific to exon6, 7 and 9 (FIG. 2B). Exons 7 in both APP and APLP2 contain the Kunitz protease inhibitor domain that is likely to mediate the angiogenic, metastatic [59, 63, 80-85] and proliferative effects of APP and APLP2 [63, 86, 87]. However, analysis of breast cancer cell lines has shown that this exon may not always be included in the cancer-type variant of APLP2 [94]. The splicing pattern of APP and APLP2 is consistent with a role of cancer-specific splicing programs [93, 95, 96]. Intriguingly, the role of Fe-dependent splicing pattern is suggested through the involvement in APP pre-mRNA processing of Fe-sensitive factors such as ASF/SF2, PTB, and SRPK1[97-100], ELAVL and U2AF2 [101] PTBP2 [102], PTBP1 [102], RBFox1[103, 104].

Fe also regulates the stability of APP and APLP2 mRNA through iron regulatory protein 2 (IRP2) that binds to the iron regulatory elements (IRE) at the 5′ untranslated regions (UTR) of APP and APLP2 mRNAs and increases their stability when Fe is abundant [105-107] as well as factors such as hnRNP C [108, 109], nucleolin [108] and FMRP [110, 111]. These findings suggest that the APP family is closely regulated by Fe-mediated survival mechanisms that also includes FAS [28, 112-114], hepcidin [115], collagen prolyl 4-hydroxylase (C-P4H) alpha-subunit [111], and MMP-9 [116].

In accordance with the present invention, it is contemplated that the APP family mediates Pt resistance through regulation of Fe homeostasis mechanism. All the current evidence supports this view. A proposed role of APP is the induction of Fe release through the transporter FPN1[80, 81, 117-119]. In this model the soluble APP acts on the endothelial cells of the microcapillaries to induce the release of Fe, which they absorb from plasma the apical side, into the tissue through their basolateral membranes [81]. A similar model was presented for cancer cells which describes how “iron addiction” in tumors [120, 121] is managed in spite of the general anemia [122]. In this model cancer cells release substances into microenvironment that convert the surrounding capillary endothelial cells, macrophages, and stromal cells into “iron donors” [123] to increase Fe availability to tumors which they take up via TF, lipocalin-2 (Lcn-2) and CD163 [124-126].

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings.

FIG. 1A: Western blots of homogenates from Pt resistant lines 2008/C13*5.23, A2780-CP and IGROV1-CP cells compared to sensitive parental lines 2008, A2780 and IGROV1. Rabbit polyclonal antibodies against human APP (top), APLP2 (middle) and APLP1 (bottom) and peroxidase conjugated goat antirabbit secondary antibodies were used. Monoclonal antibodies against tubulin were used for lane loading.

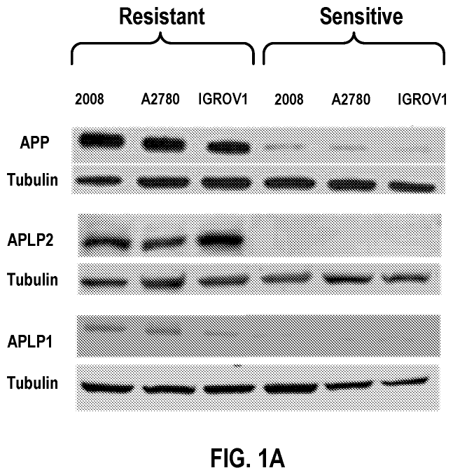

FIG. 1B: CCK-8 assay of A2780, A2780-CP and A2780-CP cells pre-incubated for 24 h with APP antisense oligonucleotides (A2780ONS) during a 1 h exposure to increasing concentrations of cDDP.

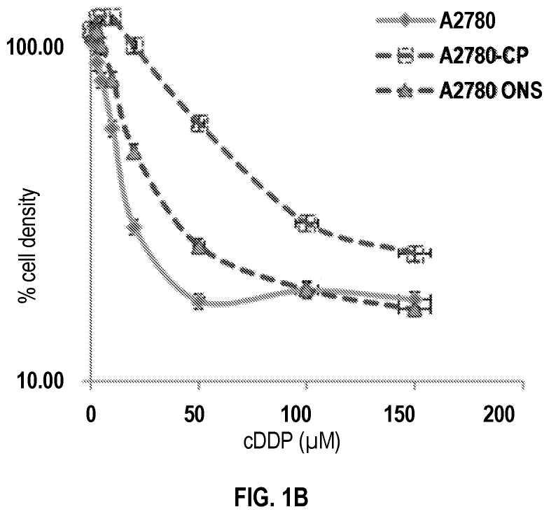

FIG. 2A: schematic view of exon-intron boundaries of the region between exons6-9 if APP pre-mRNA.

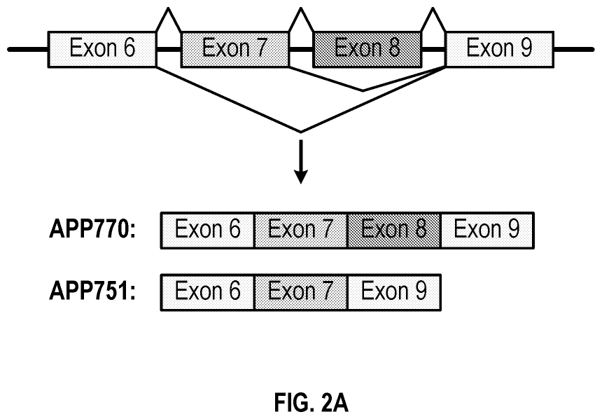

FIG. 2B: RT-PCR analysis of RNA from cDDP resistant cells 2008-C13*5.25 (1), A2780-CP (2) and IGROV-CP (3) cells using primers from the boundaries of exon 7 and exon 9 (top) and exon 7 (bottom band) after 30 cycles of amplification and resolving on 1% agarose gel.

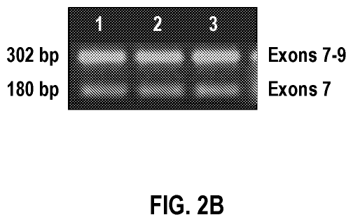

FIG. 3: A 3-dimensional model of APP; presented in ref. [2]. APP consists of E1 domain, Kunitz protease inhibitor (KPI) domain, E2 domain, transmembrane domain (TMD) and intracellular domain. The acidic region and the region between the E2 and Aβ domains are predicated to have little secondary structure

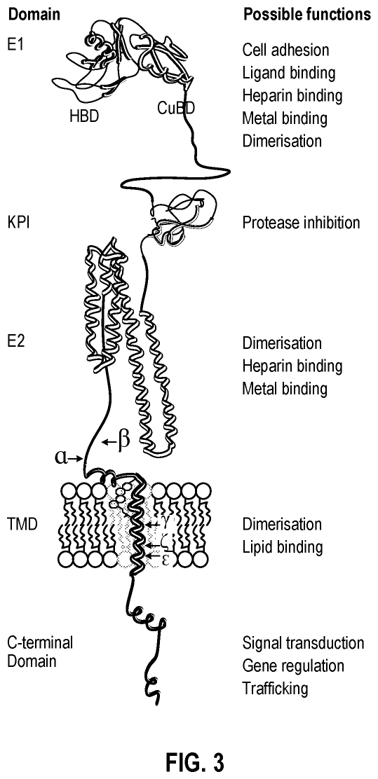

FIG. 4. The overall strategy in the production of ARPNS

FIG. 5: Model of the DR6/APP signaling complex interaction at the neuronal surface (Xu et al [1]). Binding of APP-E2 and DR6 CRD domains induces DR6 dimerization and activation. The model was generated by combining the structure reported here of the APP-E2/DR6 complex with the dimeric APP-E1 structure (Protein Data Bank ID3KTM) (Dahms et al. 2010). The two molecules in the APP dimer are colored in yellow and green, while the two DR6 molecules are colored in magenta. The linker regions in the structure were depicted as dashed lines.

### DETAILED DESCRIPTION

Provided herein are compositions comprising a DR6 peptide (-cdkc pagtyvsehc tntslrvcss cpvgtftr- SEQ ID NO:1). In particular embodiments, the composition can further comprise a conjugate comprising a DR6 peptide and a dye. In other embodiments, the conjugate can be further attached, preferably covalently, to a functionalized human serum albumin to form an ARPNS probe. In particular embodiments, either: 1-100, 1-90, 1-80, 1-70, 1-60, 1-50, 1-40, 1-30, 1-20, 1-10, 1-5, 1-2 copies of the conjugate are attached to the functionalized human serum albumin to form an ARPNS probe. In other embodiments, the number of copies of the conjugate that are attached to the functionalized human serum albumin to form an ARPNS probe can be selected from the group consisting of: 1, 2, 3, 4, 5, 6, 7, 8, 9, 10, 15, 20, 25, 30, 35, 40, 45, 50, 55, 60, 65, 70, 75, 80, 85, 90, 95 and 100.

n other embodiments, the ARPNS probe further comprises carrier molecules other than human serum albumin including natural or synthetic particles. The ARPNS probe can contain a functionalized group selected from the group consisting of anticancer drugs, antibodies well-known in the art, radioactive materials, nucleotides, dyes (e.g., fluorescent and near fluorescent, and the like) and heavy metals (e.g., molecules that can be detected by various imaging machines).

Exemplary anti-cancer drugs include: alkylating agents (e.g., cisplatin, chlorambucil, procarbazine, carmustine, and the like), antimetabolites (e.g., methotrexate, cytarabine, gemcitabine, and the like), anti-microtubule agents (e.g., vinblastine, paclitaxel, and the like), topoisomerase inhibitors (e.g., etoposide, doxorubicin, and the like), cytotoxic agents (e.g., bleomycin, mitomycin, and the like).

Exemplary anti-cancer drugs include: abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ustekinumab (Stelara), and the like.

In certain embodiments, the conjugate is attached to ARPNS probe by a cleavage site, preferably the ADAM10 cleavage site, to allow controlled release of the DR6 peptide at the tumor site. In other embodiments in may contain other cleavage sites and linkers including various enzymes and pH sensitive linkers of various sizes and qualities well-known in the art. In a particular embodiment, the DR6 peptide corresponds to: -cdkc pagtyvsehc tntslrvcss cpvgtftr—(SEQ ID NO:1).

Also provided herein are methods for treating cancer and/or a tumor, including a platinum drug resistant tumor or cancer, in a patient in need thereof, said method comprising administering and effective amount of an invention composition provided herein. In particular embodiments, the cancer is selected from the group consisting of: pancreatic, colon, breast, prostate, lung, thyroid, testicular, leukemias, lymphomas, squamous cell carcinomas, nasopharyngeal carcinomas, and oral squamous cell carcinomas.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

The unique role of APP in survival, Fe homeostasis, and Pt resistance, its absence from non-neural tissues, its tumor specific splice variants added to the well characterized properties of this molecule, supports APP as a candidate for diagnostic and therapeutic exploitation. Based on the interactions of APP with its many partners, we designed the ADR6N1probe (or APP-binding molecule) for in vivo targeting of APP-expressing Pt resistant tumor cells. In accordance with the present invention, it has been found that the ADR6N1 probe can recognize APP in vivo and that its validation as a in vivo tracer of the Pt resistant ovarian tumor will provide a much needed tool for detection, monitoring treatment of Pt resistant ovarian tumors [6].

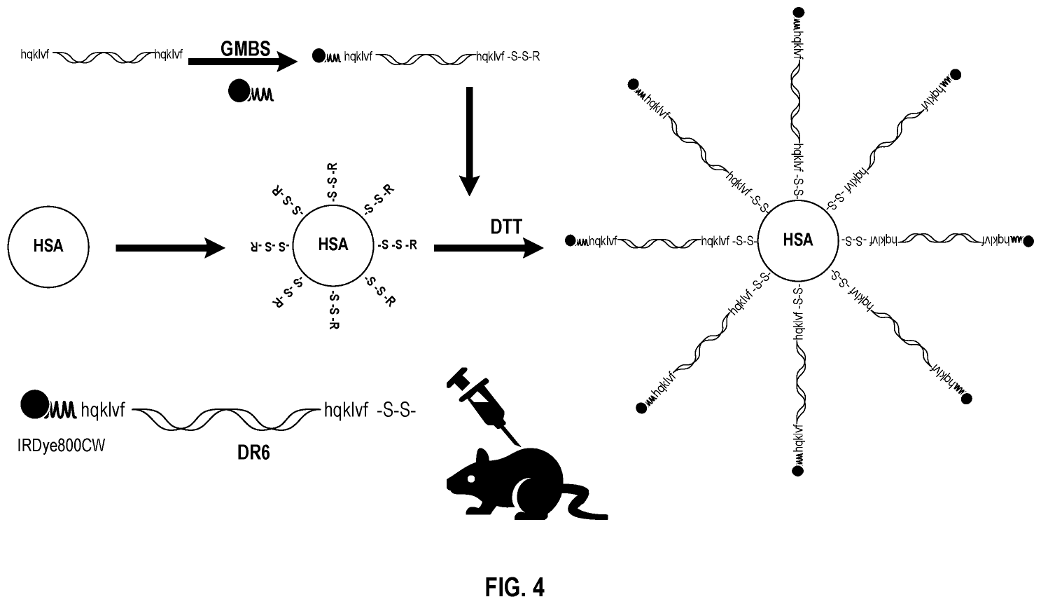

Design of ARPNS Probe. The overall strategy for the construction of the ARPNS probe is described in FIG. 4. The goal in this strategy is to construct a probe that contains many copies of the tumor detection peptide and the dye attached covalently to a carrier. We identified a 3.5 KDa peptide from the extracellular domain of the DR6/TNFRSF21 protein; the peptide binds APP with high affinity both in vivo and in vitro. To allow in vivo detection we will conjugate the DR6 peptide with the near infrared (NIR) dye IRDye800CW. In the next step, 18-20 copies of the DR6-IRDye800CW conjugate are covalently attached to a functionalized human serum albumin (H-AS) molecule to generate the ARPNS (APP Recognition Peptide-NIR-Serum albumin) probe. The ARPNS is used for in vivo and in vitro detection of APP expressing cells. The size of ARPNS is relatively small (expected to be ˜12-15 nm), which is suitable for deep penetration into tumor by Enhanced Permeation and Retention (EPR) [127, 128]. The probe is also amenable to addition of other functional groups, such as, for example, those functional groups selected from the group consisting of: anticancer drugs [129-131], antibodies [132-135], radioactive materials [136-139], and nucleotides. In particular embodiments, the ADAM10 cleavage site is added to the probe to allow controlled release of the peptide at the tumor site.

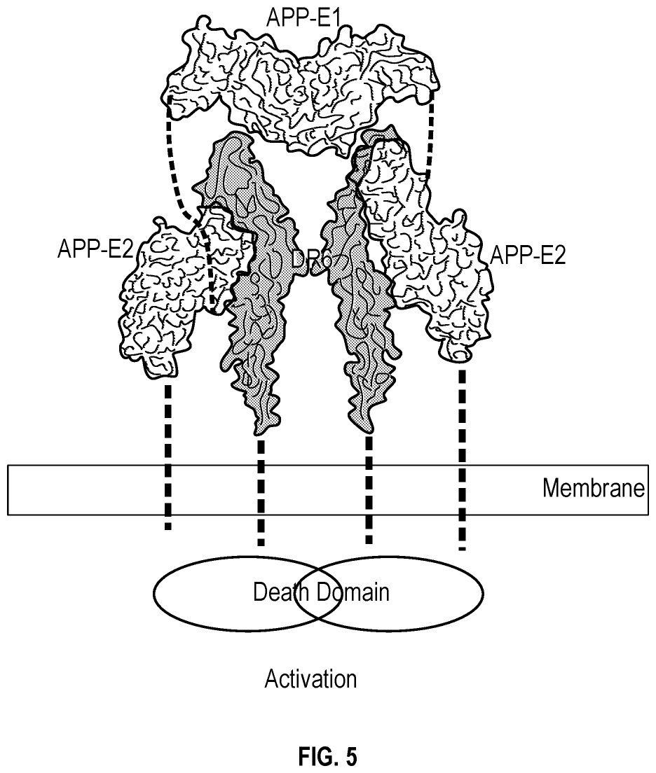

The DR6 peptide corresponding to amino acids -cdkc pagtyvsehc tntslrvcss cpvgtftr- (SEQ ID NO:1) is a 3.5 KDa fragment of the extracellular domain of the death receptor protein DR6; this domain was shown to bind APP in vivo and in vitro [1, 140, 141]. The peptide spans the (CRD1)f domain of DR6, is a member of the tumor necrosis factor receptor family. The protein can be induced by ROS via NFKB [142] and is a candidate biomarker of ovarian [143, 144], melanoma [145] and sarcoma [146] cancers. DR6 functions in metastasis [147], tumor induced necrosis [148, 149], and caspase 3-mediated apoptosis [140]. DR6 binding region in APP is within he E2 domain [1, 140, 141]. The binding may trigger events that range from pathfinding to cell death [140, 150] (FIG. 5).

While many other proteins bind APP [151] and the binding of several proteins such as F-spondin [152], SLIT1, SLIT2 [153], contactins 2, 3, 4 [151, 154] has been well documented, the DR6 interaction with APP is one of the best characterized, including the identities of the interacting amino acid residues on both proteins [1]. In addition, DR6 does not seem to bind any other extracellular or membrane protein, thus provides the specificity that we need for targeting APP expressing proteins. Aβ binding peptides such as affibodies [155] and anticalins [156] are also an option, but most of these peptides are specific to the aggregated Aβ peptides.

NIR fluorophore IRDye800CW is near infrared dye that is commonly used for in vivo imaging. The dye is attached to the C-terminus of the DR6 peptide and can be cleaved by the ADAM10 protease.

Human serum albumin (H-SA) is labeled with Alexa Fluor 633 dye on its single cysteine 34 to allow tracing of the stability of the conjugates; H-SA used as a carrier will allow attachment of multiple copies of the DR6-dye conjugate as well as serve to protect the probe from proteolysis in plasma or engulfment by phagocytes; as a carrier HSA is ideal as a natural, non-toxic and non-immunogenic protein.

Encapsulation with polyethylene glycol is considered if the probe becomes immunogenic or is endocytosed by tissue and blood macrophages.

### EXAMPLES

### Materials and Methods

The objective of this initial study is to assess the feasibility of using ARPNS as an in vivo probe for Pt resistant cells that express the APP biomarker. The use of DR6 peptide as a binding partner of APP will provide another layer of specificity, because it does not have any other extracellular partners; the provision of ADAM10 peptide at the flanking regions of the DR6 peptide will ensure the release of the peptide and the dye at the tumor site. The ADAM10 is highly expressed in A2780 cells as our Western blotting data indicates.

For binding studies, we have made extensive use of the A2780 cell line, which we have characterized in different experimental settings. This cell line was used to produce the A2780-APP-LUC and A2780-LUC sublines by introducing lentiviral constructs of a full length APP751 cDNA-tagged with the luciferase peptide and the the luciferase tag alone using pLenti CMV Puro LUC (Addgene Watertown, Mass.).

### Example 1: Demonstrate the Specify of ARPNS for Pt Drug Resistant Tumors Cells In Vitro.

In this example we will first (1) assemble the ARPNS probe and determine its physicochemical properties; and then (2) assay binding between ARPNS and purified ectodomain of APP, using ELISA assay, and between APP and ARPNS using cell surface binding assay, using A2780-APP-LUC and A2780-LUC cells. The A2780-APP-LUC and A2780-LUC is used to (3) assay the cytotoxicity of ARPNS; and (4) the cellular uptake of ARPNS.

(1) Assembly of the ARPNS probe and determination of its physicochemical properties: The functional moiety in ARPNS is the DR6 peptide; this peptide has no known post-translational modification except for N82 glycosylation that appears to fall outside of its binding domain with APP [1]. to achieve correct pos-translational modification of the peptide we will express the peptide in Sf9 cells using a baculoviral system.

(1a) Expression of DR6 peptide (corresponding to -cdkc pagtyvsehc tntslrvcss cpvgtftr- (SEQ ID NO:1)): A 96 base pair DNA fragment of DR6 cDNA that corresponds to the amino terminal sequences between residues Cys67-Arg98 has been cloned in the pTOPO-TA vector. After PCR amplification of this sequence and addition, on each of its N and C terminal ends, of the hqklvf peptide motif for ADAM10 cleavage site (found in APP ectodomain) [157] the sequence is cloned in the baculoviral vector HBM TOPO and expressed as a secreted protein in Sf9 cells (Thermo Fisher Scientific, Carlsbad, Calif.). The secreted His-tagged peptide is purified on Ni columns and excised by TEV [158] and after HPLC purification [159], it is conjugated and linked with the NIR dye IRDye800CW (LiCor Biosciences, Lincoln, Nebr.).

(1b) Attachment of DR6- peptide to IRDye800CW. The DR6 is first reacted with the GMBS (N-γ-maleimidobutyryl-oxysuccinimide ester) and then reacted with the IRDye800CW-MAL on a 1:1 ration. The construct is column purified and conjugated with the human serum albumin (H-SA) (Sigma-Aldrich, St. Louis Mo.).

(1c) Attachment of the DR6-peotide-NIR-dye conjugate to H-SA. H-SA is first reacted with the bifunctional liner succinimidyl 3-(2-pyridyldithio) propionate (SPDP) at a ratio of 1:25 molar ratio of protein/linker, it is then purified and the number of linked SPDP conjugates determined. The H-SA-SPDP is linked to DR6-thiol group of IRDye800CW at a 1:20 molar ratio of H-SA to DR6-IRDye800CW. Linkers and reagents are from Thermo Fisher Scientific, Carlsbad, Calif.).

(1d) Determination of the physicochemical properties of ARPNS in vitro. The probe is purified by HPLC and its size and molecular weight determined; the size of the probe will also be confirmed along with ζ potential determined using a Zetasizer Nano (Malvern Instruments, Westborough, Mass., USA) at 25° C.

(1e) Assessing the stability of ARPNS. Dialysis PBS plus 20% bovine serum is performed for 4-12 h at 37° C. and samples from both inside the dialysis bag and the chamber are taken in regular intervals to assess the fluorescence of NIR with a UV-3600 Plus UV-Vis-NIR Spectrophotometer.

(2) Assessment of the specificity of interaction between ARPNS and the ectodomain pf APP using ELISA and cell surface binding assays.

(2a) Assessment of the specificity of interaction between ARPNS and APP using ELISA assays. These experiments are performed in 96 well plates coated with the purified ectodomain of APP [150]. Binding is performed for 24 h at 4° C. after a 2 h blocking with PBS plus 1% BSA. Serial dilutions of ARPNS are used for incubation. Detection of the bound probe is made after incubation for 1 h with an HRP conjugated anti-DR6 polyclonal antibodies (R&D systems, Minneapolis, Minn.) and then treatment with HRP color reagents (R&D Systems) followed by determining the signal at 450 nm with a Tecan plate reader.

(2b) Assessment of the specificity of interaction between ARPNS APP on the cell surfaces. This assay is performed using cell lines A2780-APP-LUC and A2780-LUC. Cells are incubated with different dilutions of ARPNS in binding buffer with 2% BSA for 2 h on ice, and 20 min in 37oC chamber. Cells are then fixed with 3% formaldehyde and reacted with fluorescein conjugated polyclonal antibodies against DR6 (AF144, R&D Systems Minneapolis, Minn.) for 1 h and then washed and the levels of fluorescence determination by a Tecan plate reader [140, 150].

(2c) Assessment of cytotoxicity. One day after seeding A2780-APP-LUC and A2780-LUC cells in 96-well plates at 8×104 cells/well, cells are treated with different dilutions of ARPNS for 5 days after which they are assayed with Count Kit 8 (CCK-8) (Dojindo, Gaithersburg, Md.) reagents using absorption at 450 nm and a Tecan plate reader [160].

(2d) Assessment of cellular uptake: A2780-APP-LUC and A2780-LUC cells are incubated with appropriate dilutions of ARPNS for 1-4 h after which they are fixed with 3% formaldehyde and stained with antibodies against APP, DR6, Rab5, Rab7 (for endosome) and lysotracker red (lysosome) Hoechst 33342 dye (for nuclei) and then reacted with secondary fluorescent antibodies [161]. Cells are imaged with a deconvoluting confocal microscope.

The results of Example 1 indication that ADR6NI is suitable as an in vivo indicator of Pt drug resistance.

### Example 2: Demonstrate the Specificity ARPNS as an In Vivo Targeting Tool for Pt Drugs Resistant Tumors Using a Xenograft Mouse Model.

These experiments begin upon optimizing the binding of APP to ARPNS and adjusting the ARPNS probe, the A2780-APP-LUC and A2780-LUC cells to achieve maximum specificity. All animal studies are performed with the approval of the Animal Research Committee and according to the institutional guidelines at the Explora BioLabs, San Diego, Calif., where the mouse is housed. We will (1) establish mouse xenograft model using the intraperitoneal inoculation of A2780-APP-LUC and A2780-LUC cells; (2) select a suitable route of delivery for the ARPNS probe; and (3) assay binding between ARPNS and APP in vivo, using in vivo imaging, histological and biochemical analysis. Nu/Nu mice are immune compromised and are ideal for both tumor growth as well as the integrity of the probe that is less likely to be taken up by defective macrophages in these animals.

(1) Establishment of mouse xenograft model; 5-6-weeks old female Nu/Nu mice (Charles River, San Diego, Calif.) are inoculated in the peritoneal cavity with 2X106 of either A2780-APP-LUC or A2780-LUC and tumor growth is monitored with an IVIS 200 imager (Caliper Life Sciences) on isoflurane anesthetized mice. After 3 or 4 days, mice with engrafted tumor are randomly divided into control (receiving saline) and experimental groups (receiving ARPNS). Injections begin when tumors reach about 120 mm3 in diameter. The use of A2780-LUC cells is to test for non-specific binding of the probe; if the initial experiments show no in vivo association between these cells and the probe, we will exclude this set from the experiment and focus on characterizing the binding of A2780-APP-LUC xenografts with the probe in detail.

(2) Selection of route of ARPNS delivery. HSA conjugates are usually suitable for both intravenous (IV) and intraperitoneal injection (IP); to assess the advantages and disinvites of of IV vs. IP for the case of ARPNS, a single injection of 100 mg/Kg of body weight is used. Animals are imaged post-injection at 1, 3, 6 and 24 h and sacrificed at 24 h. The route of injection that produces the highest level of drug in tumors, and the lowest in different organs is used.

(3) Determination of maximum tolerated doses (MTD, is defined as the maximum dose of a drug that does not cause death or >20% body weight loss). MTF is determined by dose escalation from 25, 50, and 150 mg/Kg probe after 4 weeks of daily injection with 25, 50 and 150 mg of ARPNS/Kg body weight.

(4) Analysis of peritoneal cavity washes. After sacrifice, the peritoneal cavity is rinsed twice with PBS and the fluid collected in EDTA tubes; trypan blue cell counts and FACS analysis are performed to determine the number of necrotic and sloughed tumor cells and ARPNS levels in the cavity; the data is examined by a licensed veterinary pathologist at Histology and Comparative Pathology Shared Resource at UCSD.

(5) Terminal tissue collection and measurements of tumor burden. After sacrifice, tumors are removed, and measured for weight and volume. Blood samples are collected by cardiac puncture and blood smears prepared for testing with the Siemens Advia™120 hematology analyzer Idexx for General Panel (clinical blood chemistry and biochemistry); lung, heart, liver, spleen, kidneys, ovaries, uterus and brain are removed, weighed, assessed for damage, and their NIR and DR6 contents determined by spectrophotometry and Western blotting respectively; tissues are stored in liquid nitrogen.

(6) Histology of xenografts. All histology work is performed at the Histology and Comparative Pathology Shared Resource at UCSD. Tumors and other tissues (to be determined depending on distribution data) are fixed in 10% formalin, embedded in paraffin and sectioned at 5 μm thickness. Routine staining is with eosin hematoxylin. A board-certified veterinary pathologist will determine the degrees of vascularization, necrosis, number and type of macrophage, neutrophil, lymphocytes and eosinophils in tumors. Interaction of ARPNS with APP is determined with staining tissue sections with antibodies against APP and DR6 and localization of the NIR fluorescence.

### Statistical Analysis

All in vitro experiments are repeated at least 3 times with 3 or more samples per data point; animal studies is performed on 3-6 animals per condition. Data is presented as mean±standard error of mean (SEM). Test of significance with be with Student's t-test with significance level set at P<0.05. Analysis of variance for two or more groups is by ANOVA. GraphPad Prism4 program (GraphPad Software, San Diego, Calif.) is used for calculations. Pharmacokinetic analysis will use the WinNonlin software, version 6.2 (Pharsight Corp., Sunnyvale, Calif.).

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. Xu, K., Olsen, O., Tzvetkova-Robev, D., Tessier-Lavigne, M., and Nikolov, D. B. (2015). The crystal structure of DR6 in complex with the amyloid precursor protein provides insight into death receptor activation. Genes Dev 29, 785-790.
2. Dawkins, E., and Small, D. H. (2014). Insights into the physiological function of the beta-amyloid precursor protein: beyond Alzheimer's disease. J Neurochem 129, 756-769.
3. Nolen, B. M., and Lokshin, A. E. (2013). Biomarker testing for ovarian cancer: clinical utility of multiplex assays. Mol Diagn Ther 17, 139-146.
4. Kavanagh, J., Tresukosol, D., Edwards, C., Freedman, R., Gonzalez de Leon, C., Fishman, A., Mante, R., Hord, M., and Kudelka, A. (1995). Carboplatin reinduction after taxane in patients with platinum-refractory epithelial ovarian cancer. J Clin Oncol 13, 1584-1588.
5. Markman, M., and Bookman, M. A. (2000). Second-line treatment of ovarian cancer. Oncologist 5, 26-35.
6. Della Pepa, C., Tonini, G., Pisano, C., Di Napoli, M., Cecere, S. C., Tambaro, R., Facchini, G., and Pignata, S. (2015). Ovarian cancer standard of care: are there real alternatives? Chin J Cancer 34, 17-27.
7. Parmar, M. K., Ledermann, J. A., Colombo, N., du Bois, A., Delaloye, J. F., Kristensen, G. B., Wheeler, S., Swart, A. M., Qian, W., Torri, V., et al. (2003). Paclitaxel plus platinum-based chemotherapy versus conventional platinum-based chemotherapy in women with relapsed ovarian cancer: the ICON4/AGO-OVAR-2.2 trial. Lancet 361, 2099-2106.
8. Romero, I., and Bast, R. C., Jr. (2012). Minireview: human ovarian cancer: biology, current management, and paths to personalizing therapy. Endocrinology 153, 1593-1602.
9. Holmes, D. (2015). The problem with platinum. Nature 527, S218-219.
10. Oliver, T. G., Mercer, K. L., Sayles, L. C., Burke, J. R., Mendus, D., Lovejoy, K. S., Cheng, M. H., Subramanian, A., Mu, D., Powers, S., et al. (2010). Chronic cisplatin treatment promotes enhanced damage repair and tumor progression in a mouse model of lung cancer. Genes Dev 24, 837-852.
11. Srivastava, A. K., Han, C., Zhao, R., Cui, T., Dai, Y., Mao, C., Zhao, W., Zhang, X., Yu, J., and Wang, Q. E. (2015). Enhanced expression of DNA polymerase eta contributes to cisplatin resistance of ovarian cancer stem cells. Proc Natl Acad Sci USA 112, 4411-4416.
12. Gately, D. P., and Howell, S. B. (1993). Cellular accumulation of the anticancer agent cisplatin: a review. Br J Cancer 67, 1171-1176.
13. Howell, S. B., Safaei, R., Larson, C. A., and Sailor, M. J. (2010). Copper transporters and the cellular pharmacology of the platinum-containing cancer drugs. Mol Pharmacol 77, 887-894.
14. Safaei, R. (2006). Role of copper transporters in the uptake and efflux of platinum containing drugs. Cancer Lett 234, 34-39.
15. Chekhun, V. F., Lukyanova, N. Y., Burlaka, C. A., Bezdenezhnykh, N. A., Shpyleva, S. I., Tryndyak, V. P., Beland, F. A., and Pogribny, I. P. (2013). Iron metabolism disturbances in the MCF-7 human breast cancer cells with acquired resistance to doxorubicin and cisplatin. Int J Oncol 43, 1481-1486.
16. Wu, J., Bao, L., Zhang, Z., and Yi, X. (2017). Nrf2 induces cisplatin resistance via suppressing the iron export related gene SLC40A1 in ovarian cancer cells. Oncotarget 8, 93502-93515.
17. Naredi, P., Heath, D. D., Enns, R. E., and Howell, S. B. (1995). Cross-resistance between cisplatin, antimony potassium tartrate, and arsenite in human tumor cells. J Clin Invest 95, 1193-1198.
18. Katano, K., Kondo, A., Safaei, R., Holzer, A., Samimi, G., Mishima, M., Kuo, Y. M., Rochdi, M., and Howell, S. B. (2002). Acquisition of resistance to cisplatin is accompanied by changes in the cellular pharmacology of copper. Cancer Res 62, 6559-6565.
19. Safaei, R., Katano, K., Samimi, G., Naerdemann, W., Stevenson, J. L., Rochdi, M., and Howell, S. B. (2004). Cross-resistance to cisplatin in cells with acquired resistance to copper. Cancer Chemother Pharmacol 53, 239-246.
20. Samimi, G., Safaei, R., Katano, K., Holzer, A. K., Rochdi, M., Tomioka, M., Goodman, M., and Howell, S. B. (2004). Increased expression of the copper efflux transporter ATP7A mediates resistance to cisplatin, carboplatin, and oxaliplatin in ovarian cancer cells. Clin Cancer Res 10, 4661-4669.
21. Safaei, R., Adams, P. L., Mathews, R. A., Manorek, G., and Howell, S. B. (2013). The role of metal binding and phosphorylation domains in the regulation of cisplatin-induced trafficking of ATP7B. Metallomics 5, 964-972.
22. Collins, J. F., Hua, P., Lu, Y., and Ranganathan, P. N. (2009). Alternative splicing of the Menkes copper Atpase (Atp7a) transcript in the rat intestinal epithelium. Am J Physiol Gastrointest Liver Physiol 297, G695-707.
23. Lui, G. Y., Kovacevic, Z., Richardson, V., Merlot, A. M., Kalinowski, D. S., and Richardson, D. R. (2015). Targeting cancer by binding iron: Dissecting cellular signaling pathways. Oncotarget 6, 18748-18779.
24. Miyamoto, T., Kashima, H., Yamada, Y., Kobara, H., Asaka, R., Ando, H., Higuchi, S., Ida, K., Mvunta, D. H., and Shiozawa, T. (2016). Lipocalin 2 Enhances Migration and Resistance against Cisplatin in Endometrial Carcinoma Cells. PLoS One 11, e0155220.
25. Rychtarcikova, Z., Lettlova, S., Tomkova, V., Korenkova, V., Langerova, L., Simonova, E., Zjablovskaja, P., Alberich-Jorda, M., Neuzil, J., and Truksa, J. (2017). Tumor-initiating cells of breast and prostate origin show alterations in the expression of genes related to iron metabolism. Oncotarget 8, 6376-6398.
26. Miyazawa, M., Bogdan, A. R., and Tsuji, Y. (2018). Perturbation of Iron Metabolism by Cisplatin through Inhibition of Iron Regulatory Protein 2. Cell Chem Biol.
27. Denoyer, D., Masaldan, S., La Fontaine, S., and Cater, M. A. (2015). Targeting copper in cancer therapy: ‘Copper That Cancer’. Metallomics 7, 1459-1476.
28. Hentze, M. W., Muckenthaler, M. U., and Andrews, N. C. (2004). Balancing acts: molecular control of mammalian iron metabolism. Cell 117, 285-297.
29. Lieu, P. T., Heiskala, M., Peterson, P. A., and Yang, Y. (2001). The roles of iron in health and disease. Mol Aspects Med 22, 1-87.
30. Nyholm, S., Mann, G. J., Johansson, A. G., Bergeron, R. J., Graslund, A., and Thelander, L. (1993). Role of ribonucleotide reductase in inhibition of mammalian cell growth by potent iron chelators. J Biol Chem 268, 26200-26205.
31. Thelander, L., Graslund, A., and Thelander, M. (1983). Continual presence of oxygen and iron required for mammalian ribonucleotide reduction: possible regulation mechanism. Biochem Biophys Res Commun 110, 859-865.
32. Meyer, C. F., Wang, X., Chang, C., Templeton, D., and Tan, T. H. (1996). Interaction between c-Rel and the mitogen-activated protein kinase kinase kinase 1 signaling cascade in mediating kappaB enhancer activation. J Biol Chem 271, 8971-8976.
33. Nemoto, S., DiDonato, J. A., and Lin, A. (1998). Coordinate regulation of IkappaB kinases by mitogen-activated protein kinase kinase kinase 1 and NF-kappaB-inducing kinase. Mol Cell Biol 18, 7336-7343.
34. Bao, L. J., Jaramillo, M. C., Zhang, Z. B., Zheng, Y. X., Yao, M., Zhang, D. D., and Yi, X. F. (2014). Nrf2 induces cisplatin resistance through activation of autophagy in ovarian carcinoma. Int J Clin Exp Pathol 7, 1502-1513.
35. Cross, J. V., and Templeton, D. J. (2004). Oxidative stress inhibits MEKK1 by site-specific glutathionylation in the ATP-binding domain. Biochem J 381, 675-683.
36. Anselmo, A. N., and Cobb, M. H. (2004). Protein kinase function and glutathionylation. Biochem J 381, e1-2.
37. Kamat, P. K., Kalani, A., Rai, S., Swarnkar, S., Tota, S., Nath, C., and Tyagi, N. (2016). Mechanism of Oxidative Stress and Synapse Dysfunction in the Pathogenesis of Alzheimer's Disease: Understanding the Therapeutics Strategies. Mol Neurobiol 53, 648-661.
38. Yamamoto, T., Tsigelny, I. F., Gotz, A. W., and Howell, S. B. (2015). Cisplatin inhibits MEK1/2. Oncotarget 6, 23510-23522.
39. Ohrvik, H., Logeman, B., Turk, B., Reinheckel, T., and Thiele, D. J. (2016). Cathepsin Protease Controls Copper and Cisplatin Accumulation via Cleavage of the Ctrl Metal-binding Ectodomain. J Biol Chem 291, 13905-13916.
40. Benedikter, B. J., Weseler, A. R., Wouters, E. F. M., Savelkoul, P. H. M., Rohde, G. G. U., and Stassen, F. R. M. (2018). Redox-dependent thiol modifications: implications for the release of extracellular vesicles. Cell Mol Life Sci 75, 2321-2337.
41. Kumari, S., Badana, A. K., G, M. M., G, S., and Malla, R. (2018). Reactive Oxygen Species: A Key Constituent in Cancer Survival. Biomark Insights 13, 1177271918755391.
42. Kirkpatrick, D. L., and Powis, G. (2017). Clinically Evaluated Cancer Drugs Inhibiting Redox Signaling. Antioxid Redox Signal 26, 262-273.
43. Turski, M. L., Brady, D. C., Kim, H. J., Kim, B. E., Nose, Y., Counter, C. M., Winge, D. R., and Thiele, D. J. (2012). A novel role for copper in Ras/mitogen-activated protein kinase signaling. Mol Cell Biol 32, 1284-1295.
44. Brady, D. C., Crowe, M. S., Turski, M. L., Hobbs, G. A., Yao, X., Chaikuad, A., Knapp, S., Xiao, K., Campbell, S. L., Thiele, D. J., et al. (2014). Copper is required for oncogenic BRAF signalling and tumorigenesis. Nature 509, 492-496.
45. Haremaki, T., Fraser, S. T., Kuo, Y. M., Baron, M. H., and Weinstein, D. C. (2007). Vertebrate Ctr1 coordinates morphogenesis and progenitor cell fate and regulates embryonic stem cell differentiation. Proc Natl Acad Sci USA 104, 12029-12034.
46. Tsai, C. Y., Finley, J. C., Ali, S. S., Patel, H. H., and Howell, S. B. (2012). Copper influx transporter 1 is required for FGF, PDGF and EGF-induced MAPK signaling. Biochem Pharmacol 84, 1007-1013.
47. Richardson, D. R., Tran, E. H., and Ponka, P. (1995). The potential of iron chelators of the pyridoxal isonicotinoyl hydrazone class as effective antiproliferative agents. Blood 86, 4295-4306.
48. Chisholm, C. L., Wang, H., Wong, A. H., Vazquez-Ortiz, G., Chen, W., Xu, X., and Deng, C. X. (2016). Ammonium tetrathiomolybdate treatment targets the copper transporter ATP7A and enhances sensitivity of breast cancer to cisplatin. Oncotarget 7, 84439-84452.
49. Akladios, F. N., Andrew, S. D., and Parkinson, C. J. (2016). Investigation of the cytotoxic implications of metal chelators against melanoma and approaches to improve the cytotoxicity profiles of metal coordinating agents. Biometals 29, 789-805.
50. Liang, Z. D., Long, Y., Tsai, W. B., Fu, S., Kurzrock, R., Gagea-lurascu, M., Zhang, F., Chen, H. H., Hennessy, B. T., Mills, G. B., et al. (2012). Mechanistic basis for overcoming platinum resistance using copper chelating agents. Mol Cancer Ther 11, 2483-2494.
51. Chen, T., Li, M., Zhang, R., and Wang, H. (2009). Dihydroartemisinin induces apoptosis and sensitizes human ovarian cancer cells to carboplatin therapy. J Cell Mol Med 13, 1358-1370.
52. Wang, H., Li, M., Rinehart, J. J., and Zhang, R. (2004). Pretreatment with dexamethasone increases antitumor activity of carboplatin and gemcitabine in mice bearing human cancer xenografts: in vivo activity, pharmacokinetics, and clinical implications for cancer chemotherapy. Clin Cancer Res 10, 1633-1644.
53. Mu, Q., Yu, J., McConnachie, L. A., Kraft, J. C., Gao, Y., Gulati, G. K., and Ho, R. J. Y. (2018). Translation of combination nanodrugs into nanomedicines: lessons learned and future outlook. J Drug Target 26, 435-447.
54. Wang, M., Ma, X., Wang, J., Wang, L., and Wang, Y. (2014). Pretreatment with the gamma-secretase inhibitor DAPT sensitizes drug-resistant ovarian cancer cells to cisplatin by downregulation of Notch signaling. Int J Oncol 44, 1401-1409.
55. Chen, X., Gong, L., Ou, R., Zheng, Z., Chen, J., Xie, F., Huang, X., Qiu, J., Zhang, W., Jiang, Q., et al. (2016). Sequential combination therapy of ovarian cancer with cisplatin and gamma-secretase inhibitor MK-0752. Gynecol Oncol 140, 537-544.
56. Lee, J. H., Kim, J. Y., Kim, S. Y., Choi, S. I., Kim, K. C., Cho, E. W., and Kim, I. G. (2017). APBB1 reinforces cancer stem cell and epithelial-to-mesenchymal transition by regulating the IGF1 R signaling pathway in non-small-cell lung cancer cells. Biochem Biophys Res Commun 482, 35-42.
57. Libeu, C. A., Descamps, O., Zhang, Q., John, V., and Bredesen, D. E. (2012). Altering APP proteolysis: increasing sAPPalpha production by targeting dimerization of the APP ectodomain. PLoS One 7, e40027.
58. Hansel, D. E., Rahman, A., Wehner, S., Herzog, V., Yeo, C. J., and Maitra, A. (2003). Increased expression and processing of the Alzheimer amyloid precursor protein in pancreatic cancer may influence cellular proliferation. Cancer Res 63, 7032-7037.
59. Pandey, P., Rachagani, S., Das, S., Seshacharyulu, P., Sheinin, Y., Naslaysky, N., Pan, Z., Smith, B. L., Peters, H. L., Radhakrishnan, P., et al. (2015). Amyloid precursor-like protein 2 (APLP2) affects the actin cytoskeleton and increases pancreatic cancer growth and metastasis. Oncotarget 6, 2064-2075.
60. Meng, J. Y., Kataoka, H., Itoh, H., and Koono, M. (2001). Amyloid beta protein precursor is involved in the growth of human colon carcinoma cell in vitro and in vivo. Int J Cancer 92, 31-39.
61. Takagi, K., Ito, S., Miyazaki, T., Miki, Y., Shibahara, Y., Ishida, T., Watanabe, M., Inoue, S., Sasano, H., and Suzuki, T. (2013). Amyloid precursor protein in human breast cancer: an androgen-induced gene associated with cell proliferation. Cancer Sci 104, 1532-1538.
62. Lim, S., Yoo, B. K., Kim, H. S., Gilmore, H. L., Lee, Y., Lee, H. P., Kim, S. J., Letterio, J., and Lee, H. G. (2014). Amyloid-beta precursor protein promotes cell proliferation and motility of advanced breast cancer. BMC Cancer 14, 928.
63. Miyazaki, T., Ikeda, K., Horie-Inoue, K., and Inoue, S. (2014). Amyloid precursor protein regulates migration and metalloproteinase gene expression in prostate cancer cells. Biochem Biophys Res Commun 452, 828-833.
64. Tang, C. E., Guan, Y. J., Yi, B., Li, X. H., Liang, K., Zou, H. Y., Yi, H., Li, M. Y., Zhang, P. F., Li, C., et al. (2010). Identification of the amyloid beta-protein precursor and cystatin C as novel epidermal growth factor receptor regulated secretory proteins in nasopharyngeal carcinoma by proteomics. J Proteome Res 9, 6101-6111.
65. Takayama, K., Tsutsumi, S., Suzuki, T., Horie-Inoue, K., Ikeda, K., Kaneshiro, K., Fujimura, T., Kumagai, J., Urano, T., Sakaki, Y., et al. (2009). Amyloid precursor protein is a primary androgen target gene that promotes prostate cancer growth. Cancer Res 69, 137-142.
66. Itoh, H., Kataoka, H., Koita, H., Nabeshima, K., Inoue, T., Kangawa, K., and Koono, M. (1991). Establishment of a new human cancer cell line secreting protease nexin-II/amyloid beta protein precursor derived from squamous-cell carcinoma of lung. Int J Cancer 49, 436-443.
67. Misquitta-Ali, C. M., Cheng, E., O'Hanlon, D., Liu, N., McGlade, C. J., Tsao, M. S., and Blencowe, B. J. (2011). Global profiling and molecular characterization of alternative splicing events misregulated in lung cancer. Mol Cell Biol 31, 138-150.
68. Asiedu, M. K., Thomas, C. F., Jr., Dong, J., Schulte, S. C., Khadka, P., Sun, Z., Kosari, F., Jen, J., Molina, J., Vasmatzis, G., et al. (2018). Pathways Impacted by Genomic Alterations in Pulmonary Carcinoid Tumors. Clin Cancer Res 24, 1691-1704.
69. Krause, K., Karger, S., Sheu, S. Y., Aigner, T., Kursawe, R., Gimm, O., Schmid, K. W., Dralle, H., and Fuhrer, D. (2008). Evidence for a role of the amyloid precursor protein in thyroid carcinogenesis. J Endocrinol 198, 291-299.
70. Venkataramani, V., Rossner, C., Iffland, L., Schweyer, S., Tamboli, I. Y., Walter, J., Wirths, O., and Bayer, T. A. (2010). Histone deacetylase inhibitor valproic acid inhibits cancer cell proliferation via down-regulation of the alzheimer amyloid precursor protein. J Biol Chem 285, 10678-10689.
71. Maesako, Y., Uchiyama, T., and Ohno, H. (2003). Comparison of gene expression profiles of lymphoma cell lines from transformed follicular lymphoma, Burkitt's lymphoma and de novo diffuse large B-cell lymphoma. Cancer Sci 94, 774-781.
72. Yang, Z., Fan, Y., Deng, Z., Wu, B., and Zheng, Q. (2012). Amyloid precursor protein as a potential marker of malignancy and prognosis in papillary thyroid carcinoma. Oncol Lett 3, 1227-1230.
73. Venkataramani, V., Thiele, K., Behnes, C. L., Wulf, G. G., Thelen, P., Opitz, L., Salinas-Riester, G., Wirths, O., Bayer, T. A., and Schweyer, S. (2012). Amyloid precursor protein is a biomarker for transformed human pluripotent stem cells. Am J Pathol 180, 1636-1652.
74. Jiang, L., Yu, G., Meng, W., Wang, Z., Meng, F., and Ma, W. (2013). Overexpression of amyloid precursor protein in acute myeloid leukemia enhances extramedullary infiltration by MMP-2. Tumour Biol 34, 629-636.
75. Yamada, Y., Fujimura, T., Takahashi, S., Takayama, K., Urano, T., Murata, T., Obinata, D., Ouchi, Y., Homma, Y., and Inoue, S. (2013). Clinical significance of amyloid precursor protein in patients with testicular germ cell tumor. Adv Urol 2013, 348438.
76. Provenzano, M. J., Yu, L., Hitchler, M. J., Fitzgerald, M. P., Robinson, R. A., Wayne, S., Ver Meer, M., and Domann, F. E. (2007). AP-2 participates in the transcriptional control of the amyloid precursor protein (APP) gene in oral squamous cell carcinoma. Exp Mol Pathol 83, 277-282.
77. Arvidsson, Y., Andersson, E., Bergstrom, A., Andersson, M. K., Altiparmak, G., Illerskog, A. C., Ahlman, H., Lamazhapova, D., and Nilsson, O. (2008). Amyloid precursor-like protein 1 is differentially upregulated in neuroendocrine tumours of the gastrointestinal tract. Endocr Relat Cancer 15, 569-581.
78. Ko, S. Y., Lin, S. C., Chang, K. W., Wong, Y. K., Liu, C. J., Chi, C. W., and Liu, T. Y. (2004). Increased expression of amyloid precursor protein in oral squamous cell carcinoma. Int J Cancer 111, 727-732.
79. Cheng, T. C., Manorek, G., Samimi, G., Lin, X., Berry, C. C., and Howell, S. B. (2006). Identification of genes whose expression is associated with cisplatin resistance in human ovarian carcinoma cells. Cancer Chemother Pharmacol 58, 384-395.
80. Hefter, D., and Draguhn, A. (2017). APP as a Protective Factor in Acute Neuronal Insults. Front Mol Neurosci 10, 22.
81. McCarthy, R. C., Park, Y. H., and Kosman, D. J. (2014). sAPP modulates iron efflux from brain microvascular endothelial cells by stabilizing the ferrous iron exporter ferroportin. EMBO Rep 15, 809-815.
82. Shafi, O. (2016). Inverse relationship between Alzheimer's disease and cancer, and other factors contributing to Alzheimer's disease: a systematic review. BMC Neurol 16, 236.
83. Behrens, M. I., Lendon, C., and Roe, C. M. (2009). A common biological mechanism in cancer and Alzheimer's disease? Curr Alzheimer Res 6, 196-204.
84. Pandey, P., Sliker, B., Peters, H. L., Tuli, A., Herskovitz, J., Smits, K., Purohit, A., Singh, R. K., Dong, J., Batra, S. K., et al. (2016). Amyloid precursor protein and amyloid precursor-like protein 2 in cancer. Oncotarget 7, 19430-19444.
85. Lai, J. P., Chien, J. R., Moser, D. R., Staub, J. K., Aderca, I., Montoya, D. P., Matthews, T. A., Nagorney, D. M., Cunningham, J. M., Smith, D. I., et al. (2004). hSulf1 Sulfatase promotes apoptosis of hepatocellular cancer cells by decreasing heparin-binding growth factor signaling. Gastroenterology 126, 231-248.
86. Sobol, A., Galluzzo, P., Weber, M. J., Alani, S., and Bocchetta, M. (2015). Depletion of Amyloid Precursor Protein (APP) causes G0 arrest in non-small cell lung cancer (NSCLC) cells. J Cell Physiol 230, 1332-1341.
87. Sobol, A., Galluzzo, P., Liang, S., Rambo, B., Skucha, S., Weber, M. J., Alani, S., and Bocchetta, M. (2015). Amyloid precursor protein (APP) affects global protein synthesis in dividing human cells. J Cell Physiol 230, 1064-1074.
88. Coulter, D. W., Blatt, J., D'Ercole, A. J., and Moats-Staats, B. M. (2008). IGF-I receptor inhibition combined with rapamycin or temsirolimus inhibits neuroblastoma cell growth. Anticancer Res 28, 1509-1516.
89. Seccareccia, E., and Brodt, P. (2012). The role of the insulin-like growth factor-I receptor in malignancy: an update. Growth Horm IGF Res 22, 193-199.
90. Reinhard, C., Borgers, M., David, G., and De Strooper, B. (2013). Soluble amyloid-beta precursor protein binds its cell surface receptor in a cooperative fashion with glypican and syndecan proteoglycans. J Cell Sci 126, 4856-4861.
91. Gough, M., Parr-Sturgess, C., and Parkin, E. (2011). Zinc metalloproteinases and amyloid Beta-Peptide metabolism: the positive side of proteolysis in Alzheimer's disease. Biochem Res Int 2011, 721463.
92. Venables, J. P., Klinck, R., Bramard, A., Inkel, L., Dufresne-Martin, G., Koh, C., Gervais-Bird, J., Lapointe, E., Froehlich, U., Durand, M., et al. (2008). Identification of alternative splicing markers for breast cancer. Cancer Res 68, 9525-9531.
93. Klinck, R., Bramard, A., Inkel, L., Dufresne-Martin, G., Gervais-Bird, J., Madden, R., Paquet, E. R., Koh, C., Venables, J. P., Prinos, P., et al. (2008). Multiple alternative splicing markers for ovarian cancer. Cancer Res 68, 657-663.
94. Li, C., Kato, M., Shiue, L., Shively, J. E., Ares, M., Jr., and Lin, R. J. (2006). Cell type and culture condition-dependent alternative splicing in human breast cancer cells revealed by splicing-sensitive microarrays. Cancer Res 66, 1990-1999.
95. Buratti, E., Baralle, M., and Baralle, F. E. (2006). Defective splicing, disease and therapy: searching for master checkpoints in exon definition. Nucleic Acids Res 34, 3494-3510.
96. Dvinge, H., and Bradley, R. K. (2015). Widespread intron retention diversifies most cancer transcriptomes. Genome Med 7, 45.
97. Hayes, G. M., Carrigan, P. E., Dong, M., Reubi, J. C., and Miller, L. J. (2007). A novel secretin receptor splice variant potentially useful for early diagnosis of pancreatic carcinoma. Gastroenterology 133, 853-861.
98. Karni, R., de Stanchina, E., Lowe, S. W., Sinha, R., Mu, D., and Krainer, A. R. (2007). The gene encoding the splicing factor SF2/ASF is a proto-oncogene. Nat Struct Mol Biol 14, 185-193.
99. He, X., Pool, M., Darcy, K. M., Lim, S. B., Auersperg, N., Coon, J. S., and Beck, W. T. (2007). Knockdown of polypyrimidine tract-binding protein suppresses ovarian tumor cell growth and invasiveness in vitro. Oncogene 26, 4961-4968.
100. Cheung, H. C., Corley, L. J., Fuller, G. N., McCutcheon, I. E., and Cote, G. J. (2006). Polypyrimidine tract binding protein and Notch1 are independently re-expressed in glioma. Mod Pathol 19, 1034-1041.
101. Fragkouli, A., Koukouraki, P., Vlachos, I. S., Paraskevopoulou, M. D., Hatzigeorgiou, A. G., and Doxakis, E. (2017). Neuronal ELAVL proteins utilize AUF-1 as a co-partner to induce neuron-specific alternative splicing of APP. Sci Rep 7, 44507.
102. Smith, P., Al Hashimi, A., Girard, J., Delay, C., and Hebert, S. S. (2011). In vivo regulation of amyloid precursor protein neuronal splicing by microRNAs. J Neurochem 116, 240-247.
103. Alam, S., Suzuki, H., and Tsukahara, T. (2014). Alternative splicing regulation of APP exon 7 by RBFox proteins. Neurochem Int 78, 7-17.
104. Yin, X., Chen, C., Xu, T., Li, L., and Zhang, L. (2018). Tetrahydroxystilbene glucoside modulates amyloid precursor protein processing via activation of AKT-GSK3beta pathway in cells and in APP/PS1 transgenic mice. Biochem Biophys Res Commun 495, 672-678.
105. Rogers, J. T., Randall, J. D., Cahill, C. M., Eder, P. S., Huang, X., Gunshin, H., Leiter, L., McPhee, J., Sarang, S. S., Utsuki, T., et al. (2002). An iron-responsive element type II in the 5′-untranslated region of the Alzheimer's amyloid precursor protein transcript. J Biol Chem 277, 45518-45528.
106. Belaidi, A. A., Gunn, A. P., Wong, B. X., Ayton, S., Appukuttan, A. T., Roberts, B. R., Duce, J. A., and Bush, A. I. (2018). Marked Age-Related Changes in Brain Iron Homeostasis in Amyloid Protein Precursor Knockout Mice. Neurotherapeutics.
107. Beaudoin, M. E., Poirel, V. J., and Krushel, L. A. (2008). Regulating amyloid precursor protein synthesis through an internal ribosomal entry site. Nucleic Acids Res 36, 6835-6847.
108. Rajagopalan, L. E., Westmark, C. J., Jarzembowski, J. A., and Malter, J. S. (1998). hnRNP C increases amyloid precursor protein (APP) production by stabilizing APP mRNA. Nucleic Acids Res 26, 3418-3423.
109. Lee, E. K., Kim, H. H., Kuwano, Y., Abdelmohsen, K., Srikantan, S., Subaran, S. S., Gleichmann, M., Mughal, M. R., Martindale, J. L., Yang, X., et al. (2010). hnRNP C promotes APP translation by competing with FMRP for APP mRNA recruitment to P bodies. Nat Struct Mol Biol 17, 732-739.
110. Westmark, C. J., and Malter, J. S. (2007). FMRP mediates mGluR5-dependent translation of amyloid precursor protein. PLoS Biol 5, e52.
111. Fahling, M., Mrowka, R., Steege, A., Martinka, P., Persson, P. B., and Thiele, B. J. (2006). Heterogeneous nuclear ribonucleoprotein-A2/B1 modulate collagen prolyl 4-hydroxylase, alpha (I) mRNA stability. J Biol Chem 281, 9279-9286.
112. Tejedor, J. R., Papasaikas, P., and Valcarcel, J. (2015). Genome-wide identification of Fas/CD95 alternative splicing regulators reveals links with iron homeostasis. Mol Cell 57, 23-38.
113. Bird, A. J. (2015). Cellular sensing and transport of metal ions: implications in micronutrient homeostasis. J Nutr Biochem 26, 1103-1115.
114. Muckenthaler, M. U., Rivella, S., Hentze, M. W., and Galy, B. (2017). A Red Carpet for Iron Metabolism. Cell 168, 344-361.
115. Toki, Y., Sasaki, K., Tanaka, H., Yamamoto, M., Hatayama, M., Ito, S., Ikuta, K., Shindo, M., Hasebe, T., Nakajima, S., et al. (2016). A selective splicing variant of hepcidin mRNA in hepatocellular carcinoma cell lines. Biochem Biophys Res Commun 476, 501-507.
116. Gorospe, M., Tominaga, K., Wu, X., Fahling, M., and Ivan, M. (2011). Post-Transcriptional Control of the Hypoxic Response by RNA-Binding Proteins and MicroRNAs. Front Mol Neurosci 4, 7.
117. Duce, J. A., Tsatsanis, A., Cater, M. A., James, S. A., Robb, E., Wikhe, K., Leong, S. L., Perez, K., Johanssen, T., Greenough, M. A., et al. (2010). Iron-export ferroxidase activity of beta-amyloid precursor protein is inhibited by zinc in Alzheimer's disease. Cell 142, 857-867.
118. Wong, B. X., and Duce, J. A. (2014). The iron regulatory capability of the major protein participants in prevalent neurodegenerative disorders. Front Pharmacol 5, 81.
119. Konig, G., Monning, U., Czech, C., Prior, R., Banati, R., Schreiter-Gasser, U., Bauer, J., Masters, C. L., and Beyreuther, K. (1992). Identification and differential expression of a novel alternative splice isoform of the beta A4 amyloid precursor protein (APP) mRNA in leukocytes and brain microglial cells. J Biol Chem 267, 10804-10809.
120. Torti, S. V., and Torti, F. M. (2011). Ironing out cancer. Cancer Res 71, 1511-1514.
121. Torti, S. V., and Torti, F. M. (2013). Iron and cancer: more ore to be mined. Nat Rev Cancer 13, 342-355.
122. Ganz, T. (2011). Hepcidin and iron regulation, 10 years later. Blood 117, 4425-4433.
123. Liang, W., Li, Q., and Ferrara, N. (2018). Metastatic growth instructed by neutrophil-derived transferrin. Proc Natl Acad Sci U S A 115, 11060-11065.
124. Urano, S., Ohara, T., Noma, K., Katsube, R., Ninomiya, T., Tomono, Y., Tazawa, H., Kagawa, S., Shirakawa, Y., Kimura, F., et al. (2016). Iron depletion enhances the effect of sorafenib in hepatocarcinoma. Cancer Biol Ther 17, 648-656.

### 125. Jung, M., Weigert, A., Mertens, C., Rehwald, C., and Brune, B. (2017). Iron Handling in Tumor-Associated Macrophages—Is There a New Role for Lipocalin-2? Front Immunol 8, 1171.

126. Coma, G., Campana, L., Pignatti, E., Castiglioni, A., Tagliafico, E., Bosurgi, L., Campanella, A., Brunelli, S., Manfredi, A. A., Apostoli, P., et al. (2010). Polarization dictates iron handling by inflammatory and alternatively activated macrophages. Haematologica 95, 1814-1822.
127. Zhang, L., Gu, F. X., Chan, J. M., Wang, A. Z., Langer, R. S., and Farokhzad, O. C. (2008). Nanoparticles in medicine: therapeutic applications and developments. Clin Pharmacol Ther 83, 761-769.
128. McKeon, A. M., Noonan, J., Devocelle, M., Murphy, B. M., and Griffith, D. M. (2017). Platinum(iv) oxaliplatin-peptide conjugates targeting memHsp70+ phenotype in colorectal cancer cells. Chem Commun (Camb) 53, 11318-11321.
129. Shi, J., Votruba, A. R., Farokhzad, O. C., and Langer, R. (2010). Nanotechnology in drug delivery and tissue engineering: from discovery to applications. Nano Lett 10, 3223-3230.
130. Hassan, R., Miller, A. C., Sharon, E., Thomas, A., Reynolds, J. C., Ling, A., Kreitman, R. J., Miettinen, M. M., Steinberg, S. M., Fowler, D. H., et al. (2013). Major cancer regressions in mesothelioma after treatment with an anti-mesothelin immunotoxin and immune suppression. Sci Transl Med 5, 208ra147.
131. Hassan, R., Sharon, E., Thomas, A., Zhang, J., Ling, A., Miettinen, M., Kreitman, R. J., Steinberg, S. M., Hollevoet, K., and Pastan, I. (2014). Phase 1 study of the antimesothelin immunotoxin SS1 P in combination with pemetrexed and cisplatin for front-line therapy of pleural mesothelioma and correlation of tumor response with serum mesothelin, megakaryocyte potentiating factor, and cancer antigen 125. Cancer 120, 3311-3319.

### 132. Katz, J., Janik, J. E., and Younes, A. (2011). Brentuximab Vedotin (SGN-35). Clin Cancer Res 17, 6428-6436.

133. Doronina, S. O., Toki, B. E., Torgov, M. Y., Mendelsohn, B. A., Cerveny, C. G., Chace, D. F., DeBlanc, R. L., Gearing, R. P., Bovee, T. D., Siegall C. B., et al. (2003). Development of potent monoclonal antibody auristatin conjugates for cancer therapy. Nat Biotechnol 21, 778-784.
134. Verma, S., Miles, D., Gianni, L., Krop, I. E., Welslau, M., Baselga, J., Pegram, M., Oh, D. Y., Dieras, V., Guardino, E., et al. (2012). Trastuzumab emtansine for HER2-positive advanced breast cancer. N Engl J Med 367, 1783-1791.
135. Spring, B. Q., Abu-Yousif, A. O., Palanisami, A., Rizvi, I., Zheng, X., Mai, Z., Anbil, S., Sears, R. B., Mensah, L. B., Goldschmidt, R., et al. (2014). Selective treatment and monitoring of disseminated cancer micrometastases in vivo using dual-function, activatable immunoconjugates. Proc Natl Acad Sci U S A 111, E933-942.
136. Mew, D., Wat, C. K., Towers, G. H., and Levy, J. G. (1983). Photoimmunotherapy: treatment of animal tumors with tumor-specific monoclonal antibody-hematoporphyrin conjugates. J Immunol 130, 1473-1477.
137. Mitsunaga, M., Ogawa, M., Kosaka, N., Rosenblum, L. T., Choyke, P. L., and Kobayashi, H. (2011). Cancer cell-selective in vivo near infrared photoimmunotherapy targeting specific membrane molecules. Nat Med 17, 1685-1691.
138. Back, T., Chouin, N., Lindegren, S., Kahu, H., Jensen, H., Albertsson, P., and Palm, S. (2017). Cure of Human Ovarian Carcinoma Solid Xenografts by Fractionated alpha-Radioimmunotherapy with (211)At-MX35-F(ab′)2: Influence of Absorbed Tumor Dose and Effect on Long-Term Survival. J Nucl Med 58, 598-604.
139. Terwisscha van Scheltinga, A. G., Ogasawara, A., Pacheco, G., Vanderbilt, A. N., Tinianow, J. N., Gupta, N., Li, D., Firestein, R., Marik, J., Scales, S. J., et al. (2017). Preclinical Efficacy of an Antibody-Drug Conjugate Targeting Mesothelin Correlates with Quantitative 89Zr-ImmunoPET. Mol Cancer Ther 16, 134-142.
140. Nikolaev, A., McLaughlin, T., O'Leary, D. D., and Tessier-Lavigne, M. (2009). APP binds DR6 to trigger axon pruning and neuron death via distinct caspases. Nature 457, 981-989.
141. Kuester, M., Kemmerzehl, S., Dahms, S. O., Roeser, D., and Than, M. E. (2011). The crystal structure of death receptor 6 (DR6): a potential receptor of the amyloid precursor protein (APP). J Mol Biol 409, 189-201.
142. Luong, T. T. D., Tran, G. V. Q., Shin, D. J., Lim, Y. S., and Hwang, S. B. (2017). Hepatitis C Virus Exploits Death Receptor 6-mediated Signaling Pathway to Facilitate Viral Propagation. Sci Rep 7, 6445.
143. McNeal, S., Bitterman, P., Bahr, J. M., Edassery, S. L., Abramowicz, J. S., Basu, S., and Barua, A. (2016). Association of Immunosuppression with DR6 Expression during the Development and Progression of Spontaneous Ovarian Cancer in Laying Hen Model. J Immunol Res 2016, 6729379.
144. Barua, A., Edassery, S. L., McNeal, S., Bahr, J. M., Bitterman, P., Basu, S., Sharma, S., and Abramowicz, J. S. (2016). Enhancement of Ovarian Tumor Detection by DR6-Targeted Ultrasound Imaging Agents in Laying Hen Model of Spontaneous Ovarian Cancer. Int J Gynecol Cancer 26, 1375-1385.
145. Yang, X., Shi, B., Li, L., Xu, Z., Ge, Y., Shi, J., Liu, Y., and Zheng, D. (2016). Death receptor 6 (DR6) is required for mouse B16 tumor angiogenesis via the NF-kappaB, P38 MAPK and STAT3 pathways. Oncogenesis 5, e206.
146. Yang, K., Mooney, C., Spahlinger, G., Schuetze, S., Arias-Pulido, H., Verschraegen, C., Gimotty, P., and Buckanovich, R. J. (2012). DR6 as a diagnostic and predictive biomarker in adult sarcoma. PLoS One 7, e36525.
147. Shi, B., Bao, J., Liu, Y., and Shi, J. (2018). Death receptor 6 promotes ovarian cancer cell migration through KIF11. FEBS Open Bio 8, 1497-1507.
148. Strilic, B., Yang, L., Albarran-Juarez, J., Wachsmuth, L., Han, K., Muller, U. C., Pasparakis, M., and Offermanns, S. (2016). Tumour-cell-induced endothelial cell necroptosis via death receptor 6 promotes metastasis. Nature 536, 215-218.
149. Daniel, P. T., Wieder, T., Sturm, I., and Schulze-Osthoff, K. (2001). The kiss of death: promises and failures of death receptors and ligands in cancer therapy. Leukemia 15, 1022-1032.
150. Olsen, O., Kallop, D. Y., McLaughlin, T., Huntwork-Rodriguez, S., Wu, Z., Duggan, C. D., Simon, D. J., Lu, Y., Easley-Neal, C., Takeda, K., et al. (2014). Genetic analysis reveals that amyloid precursor protein and death receptor 6 function in the same pathway to control axonal pruning independent of beta-secretase. J Neurosci 34, 6438-6447.
151. Bai, Y., Markham, K., Chen, F., Weerasekera, R., Watts, J., Home, P., Wakutani, Y., Bagshaw, R., Mathews, P. M., Fraser, P. E., et al. (2008). The in vivo brain interactome of the amyloid precursor protein. Mol Cell Proteomics 7, 15-34.
152. Ho, A., and Sudhof, T. C. (2004). Binding of F-spondin to amyloid-beta precursor protein: a candidate amyloid-beta precursor protein ligand that modulates amyloid-beta precursor protein cleavage. Proc Natl Acad Sci U S A 101, 2548-2553.
153. Wang, B., Li, H., Mutlu, S. A., Bowser, D. A., Moore, M. J., Wang, M. C., and Zheng, H. (2017). The Amyloid Precursor Protein Is a Conserved Receptor for Slit to Mediate Axon Guidance. eNeuro 4.
154. Osterfield, M., Egelund, R., Young, L. M., and Flanagan, J. G. (2008). Interaction of amyloid precursor protein with contactins and NgCAM in the retinotectal system. Development 135, 1189-1199.
155. Lindgren, J., Wahlstrom, A., Danielsson, J., Markova, N., Ekblad, C., Graslund, A., Abrahmsen, L., Karlstrom, A. E., and Warmlander, S. K. (2010). N-terminal engineering of amyloid-beta-binding Affibody molecules yields improved chemical synthesis and higher binding affinity. Protein Sci 19, 2319-2329.
156. Rauth, S., Hinz, D., Borger, M., Uhrig, M., Mayhaus, M., Riemenschneider, M., and Skerra, A. (2016). High-affinity Anticalins with aggregation-blocking activity directed against the Alzheimer beta-amyloid peptide. Biochem J 473, 1563-1578.
157. Lammich, S., Kojro, E., Postina, R., Gilbert, S., Pfeiffer, R., Jasionowski, M., Haass, C., and Fahrenholz, F. (1999). Constitutive and regulated alpha-secretase cleavage of Alzheimer's amyloid precursor protein by a disintegrin metalloprotease. Proc Natl Acad Sci U S A 96, 3922-3927.
158. Klima, M., Zajedova, J., Doubravska, L., and Andera, L. (2009). Functional analysis of the posttranslational modifications of the death receptor 6. Biochim Biophys Acta 1793, 1579-1587.
159. Safaei, R., Adams, P. L., Maktabi, M. H., Mathews, R. A., and Howell, S. B. (2012). The CXXC motifs in the metal binding domains are required for ATP7B to mediate resistance to cisplatin. J Inorg Biochem 110, 8-17.
160. Larson, C. A., Adams, P. L., Blair, B. G., Safaei, R., and Howell, S. B. (2010). The role of the methionines and histidines in the transmembrane domain of mammalian copper transporter 1 in the cellular accumulation of cisplatin. Mol Pharmacol 78, 333-339.
161. Safaei, R., Larson, B. J., Cheng, T. C., Gibson, M. A., Otani, S., Naerdemann, W., and Howell, S. B. (2005). Abnormal lysosomal trafficking and enhanced exosomal export of cisplatin in drug-resistant human ovarian carcinoma cells. Mol Cancer Ther 4, 1595-1604.

1. A composition comprising a DR6 peptide.

2. A composition comprising a conjugate comprising a DR6 peptide and a dye.

3. The composition of claim 2, wherein the conjugate is further attached to a functionalized human serum albumin to form an ARPNS probe.

4. The composition of claim 2, wherein the conjugate is attached covalently to a functionalized human serum albumin, natural or synthetic particle, to form an ARPNS probe.

5. The composition of claim 4, wherein the number of copies of the conjugate that are attached to the functionalized human serum albumin, natural or synthethic particle, to form an ARPNS probe is be selected from the group consisting of: 1, 2, 3, 4, 5, 6, 7, 8, 9, 10, 15, 20, 25, 30, 35, 40, 45, 50, 55, 60, 65, 70, 75, 80, 85, 90, 95 and 100.

6. The composition of claims 3, wherein the ARPNS probe further comprises a functionalized group selected from the group consisting of: anticancer drugs, antibodies, radioactive materials, nucleotides, dyes and heavy metals.

7. The composition of claims 3, wherein the conjugate is attached to ARPNS probe by a cleavage site to allow controlled release of the DR6 peptide at the tumor site.

8. The composition of claim 7, wherein the cleavage site is the ADAM10 cleavage site.

9. The composition of claims 1, wherein the DR6 peptide is: -cdkc pagtyvsehc tntslrvcss cpvgtftr- (SEQ ID NO:1).

10. A method for treating a tumor or cancer in a patient in need thereof, said method comprising administering a therapeutically effective amount of the composition of claims 1.

11. The method of claim 10, wherein the tumor or cancer is platinum drug resistant.

12. The method of claim 10, wherein the cancer is selected from the group consisting of: pancreatic, colon, breast, prostate, lung, thyroid, testicular, leukemias, lymphomas, squamous cell carcinomas, nasopharyngeal carcinomas, and oral squamous cell carcinomas.
